(19) United States
(12) Patent Application Publication
(10) Pub. No.: US 2017/0088623 A1
(43) Pub. Date: Mar. 30, 2017

(54) Prophylaxis Against Cancer Metastasis

(71) Applicants: Eckhard U. Alt, Houston, TX (US); Ralph B. Arlinghaus, Houston, TX (US); Michael E. Coleman, Houston, TX (US)

(72) Inventors: Eckhard U. Alt, Houston, TX (US); Ralph B. Arlinghaus, Houston, TX (US); Michael E. Coleman, Houston, TX (US)

(73) Assignees: Ingeneron, Inc., Houston, TX (US); Board of Regents, The University of Texas System, Austin, TX (US)

(21) Appl. No.: 14/871,715

(22) Filed: Sep. 30, 2015

(51) Int. Cl. C07K 16/28; C07K 14/715

(52) U.S. Cl. CPC C07K 16/2866; C07K 14/7158

(57) ABSTRACT

This document provides prophylactic methods for reducing cancer metastasis by targeting LCN2, MMP9, and CX-CR4.

### CROSS REFERENCE TO RELATED APPLICATIONS

This application is a divisional of, and claims priority to, U.S. application Ser. No. 13/387,852, filed May 14, 2012, published as U.S. 2012/0219561 on Aug. 30, 2012, which claims priority as a filing under §371 from PCT/US2010/044070, filed Aug. 2, 2010 (published as WO2011/014863 on Feb. 3, 2011), which in turn claims priority to U.S. Provisional Application Ser. No. 61/230,439 filed Jul. 31, 2009, all of which are incorporated herein by reference.

### FIELD OF THE INVENTION

This invention relates generally to compositions and methods for preventing cancer metastasis. The present invention relates more particularly to selective targeting of LCN2, MMP9, and CXCR4 and prophylactic blocking of these molecules to prevent cancer metastasis.

### BACKGROUND OF THE INVENTION

Without limiting the scope of the invention, its background is described in connection with existing methods and compositions for blocking cancer metastasis. Metastasis marks the spread of cancer cells from a primary site and the establishment of distant tumors. Metastasis of cancer cells is responsible for over 90% of cancer mortality. The molecular bases of metastatic events have been investigated over many years but a mechanistic understanding remains incomplete. In addition to immune escape mechanisms, it is believed that altered cell adhesion, survival, proteolysis and tissue remodeling, migration, and homing on target organs participate in the process. Similar processes are found in embryonic development, and, to a different extent, in adult tissue maintenance and repair. A certain prerequisite for cancer metastasis is the escape of tumor cells from the primary cluster.

In certain instances, a risk of dissemination of tumor cells may be associated with a procedure or intervention. Establishment of a new site of tumor growth as a consequence of a surgical procedure is variously known as implantation metastasis, tumor seeding or malignant seeding.

For example, one procedure related complication of the aspiration biopsy of malignant tumors is dissemination of tumor cells along the needle track. Tumor seeding is also known to occur in association with surgical resection, particularly where resection is likely to yield close or positive margins. Although such risks have been largely downplayed in the scientific literature, procedure related spread of malignant cells has been documented. In hepatocellular carcinoma, reported rates of seeding along the needle track range from 0.6% to 5.1% using various conventional biopsy techniques and such seeding has been described as “a dreaded complication of percutaneous biopsy.” Maturen, KE, et al. “Lack of Tumor Seeding of Hepatocellular Carcinoma after Percutaneous Needle Biopsy Using Coaxial Cutting Needle Technique” Am. J. Roentgenology 187 (2006) 1184. Needle tract tumor seeding is a complication reported to occur at of rate of 0.5-2.8% of percutaneous radiofrequency (RF) ablation procedures for treatment of hepatic tumors. Breast cancer recurrence from tumor cells seeding the biopsy needle tract in patients after definitive surgical therapy (without adjuvant radiation therapy) has lead some to recommend excising the stereotactic core biopsy tract at the time of definitive surgical resection of the primary tumor. Chao, C. et al. “Local recurrence of breast cancer in the stereotactic core needle biopsy site: case reports and review of the literature” Breast J 7(2) (2001) 124.

In one large study of the risks of tumor dissemination by laparoscopic cholecystectomy, port-site recurrence was observed in 70 out of 409 (17%) patients in which an incidental gallbladder carcinoma was discovered in post-surgical pathology. In the case of laproscopic resection of colorectal carcinoma, tumor seeding was identified in 19 out of 412 (4.6%) patients. Finally, 14 cases of trocar site metastasis were identified after laparoscopy for different nonapparent or known malignancies. Paolucci, V., et al. “Tumor Seeding following Laparoscopy: International Study” World J of Surg. 23 (1999) 989.

Certain cancers are particularly notorious for procedure related tumor seeding. In a recent study of tumor seeding in 100 mesothelioma patients, the incidence of needle track seeding was 4% for image-guided core-needle biopsy and 22% for surgical biopsy. Agarwal, PP., et al. “Pleural Mesothelioma: Sensitivity and Incidence of Needle Track Seeding after Image-guided Biopsy versus Surgical Biopsy” Radiology 241 (2006) 589.

Tumor seeding at the stoma of a percutaneous endoscopic gastrostomy (PEG) tube is a major, albeit rare, complication in the nutritional management of oropharyngeal squamous cell carcinoma. Mincheff TV. “Metastatic spread to a percutaneous gastrostomy site from head and neck cancer; case report and literature review” J Soc. Laproendoscopic Surgeons 9 (2005) 466-471. The potential spread of previously localized tumor cells by mechanical compression during mammography has been discussed in the literature. Watmough, DJ, et al. “For Debate: Does breast cancer screening depend on a wobbly hypothesis?” J. Public Health Medicine 19 (4) (1997) 375. However rare these events may be, tumor cell dissemination by a routine diagnostic procedure is highly undesirable.

Prophylaxis for tumor seeding has been tested. Experimental modalities have included pre-procedure radiotherapy and perioperative administration of chemotherapeutic drugs. Complications of these modalities include those typically associated with such cytotoxic procedures and precludes the wide-spread adoption of prophylaxis when balanced against the belief that tumor seeding occurs in only a minority of patients.

From the foregoing it is apparent that there is a need for non-toxic prophylaxis against metastatic dissemination of cancer cells in procedures that may carry a risk of such dissemination. The present inventors have applied a mechanistic approach to the identification of compositions able to block a potential for procedure related metastatic spread.

### BRIEF SUMMARY OF THE INVENTION

The present invention is directed to a method for preventing the tumor cell metastasis and tumor seeding that potentially occurs as a consequence of a surgical or diagnostic procedure. Thus, in certain embodiments antagonists to the identified targets are administered before, during and/or after a procedure that might carry a risk of dissemination of tumor cells. Because the antagonists have very low toxicity, procedure related administration confers the benefit of blocking growth of any tumor cells that might be released without side effects.

In one particular embodiment of the invention, one or more antagonists to LCN2, LCN2/MMP9 and/or CXCR4 are utilized as prophylaxis against tumor seeding and metastasis. In preferred embodiments, a cocktail of antagonists is employed to block metastasis through multiple mechanisms. For example, antagonists to LCN2 and/or LCN2/MMP6 are combined with antagonists to CXCR4 and administered as a prophylactic against metastasis that may occur as a consequence of a procedure that allows escape of tumor cells from the primary tumor. In one embodiment the antagonists are antibodies. In one embodiment, the LCN2 specific antibody blocks the interaction of LCN2 with MMP9.

In other embodiments antagonists are generated to the covalently linked, disulfide-bridged heterodimer of LCN2 and MMP9 and the heterodimer antagonists are administered as prophylaxis against metastatic spread potentially associated with a surgical or diagnostic procedure.

In certain embodiments, a cocktail of antagonists is administered including antagonists directed to the LCN2 interaction with MMP9 formulated together with antagonists to the interaction between CXCR4 and CXCL12. The cocktail is administered before, contemporaneous with, and/or after a surgical or diagnostic procedure that carries a theoretical risk of tumor cell metastasis and tumor seeding.

### BRIEF DESCRIPTION OF THE DRAWINGS

For a more complete understanding of the present invention, including features and advantages, reference is now made to the detailed description of the invention along with the accompanying figures:

FIG. 1 illustrates that high NGAL expression correlates with decreased survival in breast cancer patients.

FIG. 2 depicts the time course of primary mammary tumor development in MMTV-ErbB2 (V664E) transgenic mice in the three genetic backgrounds: mLCN2^{+/+}, mLCN2^{+/-}, and mLCN2^{-/-}, depicted by the Kaplan-Meier analysis. T₅₀ is a calculated statistical value incorporating both time and incidence of tumor formation when 50% of the mice in the same group developed mammary tumors.

FIGS. 3A-D depict experiments showing that LCN2 (NGAL) is a down-stream target of the HER2/PI-3K/AKT/NF-κB pathway. FIG. 3A shows Western blotting of LCN2 levels in HER2⁺ SKBr3 cells treated with Herceptin. FIG. 3B shows Western blotting of LCN2 levels in SKBr3 cells incubated with PI-3K inhibitor LY 294002. FIG. 3C shows Western blotting of LCN2 levels in SKBr3 cells incubated with the NF-KB inhibitor Bay11-7082. FIG. 3D shows Western blotting of LCN2 levels in SKBr3 cells transiently expressing active or dominant negative AKT.

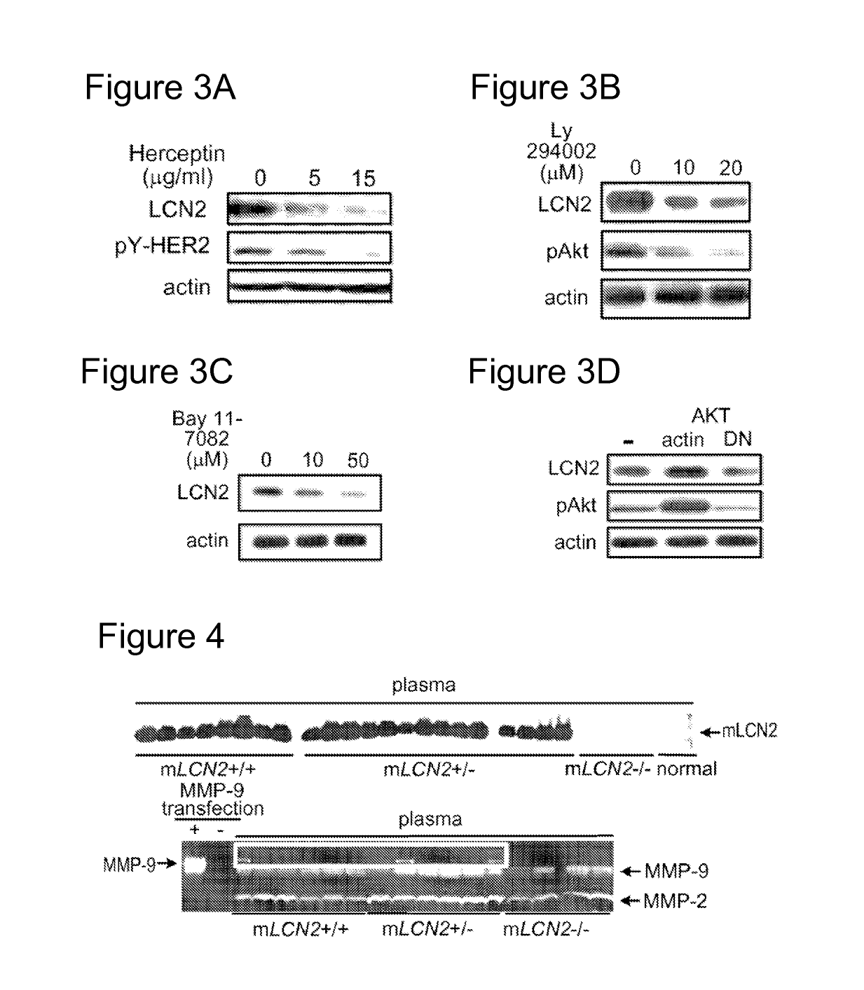

FIG. 4 shows the levels of mLCN2 (upper panel) and MMP activity (lower panel) in the plasma of the three mice groups expressing different levels of LCN2. The white box in the lower panel marks high molecular weight MMP activity.

FIGS. 5A-D show the mediators for interaction between stem cells and cancer cells. FIG. 5A shows that 4T1 cells and especially spheroid-forming 4T1 cells migrate toward the conditioned medium of mASC. 4T1 cells (3×10⁴) were plated in the upper chamber of a 3 μm transwell system. The lower chamber was filled with 1 ml of 72 h conditioned medium of mASC. After 24 h of migration through the transwell membrane, cells were fixed and stained with calcein. The results are the mean and SD number of migrated cells per microscopic field under fluorescent microscope. FIG. 5B shows data that mASC but not 4T1 cells produce SDF-1; 4T1 cells especially spheroid-forming 4T1 cells show higher messenger RNA levels of the specific receptor CXCR4. Secreted amounts of SDF-1a were measured by enzyme-linked immunosorbent assay and CXCR4 messenger RNA levels were determined by quantitative reverse transcription—polymerase chain reaction (RT—PCR). Reverse transcription—polymerase chain reaction results were normalized against GAPDH. FIG. 5C shows that 4T1 cells and mammospheres migrate toward recombinant SDF-1 in a transwell system. Recombinant murine SDF-1 in serum-free medium was filled into the lower chamber in stated concentrations. CXCR4 inhibition was performed by 30 μg/ml neutralizing antibody 1 h before and during the migration assay. FIG. 5D shows that 4T1 migration toward mASC conditioned medium is mediated mainly by SDF-1. *P<0.05, ***P<0.001, ****P<0.001.

FIG. 6A represents data showing that tumor growth is enhanced when 4T1 spheroid-forming cells are co-injected with mASC. A total of 5×10³ 4T1 spheroid-forming cells or spheroid-forming cells with CXCR4 knockdown, respectively, were injected or co-injected with 5×10⁴ GFP-labeled mASC subcutaneously. Values represent volume measurements (mm³) with scientific caliper. FIG. 6B shows tumor volume 21 days post-injection is increased when 4T1 spheroid-forming cells are co-injected with mASC and decreased using 4T1 cells with CXCR4 knockdown. Columns represent tumor volumes in mm³±SDs evaluated from microCT images at day 21.

### DETAILED DESCRIPTION OF THE INVENTION

While the making and using of various embodiments of the present disclosure are discussed in detail below, it should be appreciated that the present disclosure includes several inventive concepts which can be employed in a variety of specific contexts. The specific embodiments discussed herein are merely illustrative of specific ways to make and use the invention and do not delimit the scope of the claims.

To facilitate the understanding of the claims, a number of terms are defined below. Terms defined herein have meanings as commonly understood by a person of ordinary skill in the areas relevant to the present disclosure. Terms such as “a”, “an” and “the” are not intended to refer to only a singular entity, but include the general class of which a specific example may be used for illustration.

For purposes of the present invention, the term “LCN2” refers to lipocalin-2, also known as Neutrophil Gelatinase-Associated Lipocalin or “NGAL” in humans and 24p3 in the mouse. The reference sequence for the human mRNA is NM_005564.3, while the reference sequence for the human protein is NP_005555. The lipocalin protein family is a diverse superfamily divided into two structurally defined groups: the kernel lipocalins and the outlier lipocalins. All lipocalins feature a single eight-stranded antiparallel β-sheet closed back on itself to form a continuously hydrogen-bonded β-barrel that forms a cup shaped internal ligand-binding site. Lipocalin 2 (LCN2) is a 198 amino acid (22588 mol. weight (Da)) protein, which, when folded, contains a single di-sulfide bond. Identification of a cell surface receptor for 24p3 (mouse) lead to identification of solute carrier family 22, member 17 (SLC22A17), as the cell surface receptor for LCN2 (NGAL). Devireddy, LR et al. “A cell surface receptor for lipocalin 24p3 selectively mediates apoptosis and iron uptake” Cell 123 (7) (2005) 1293. In addition to functioning as a known transporter of small lipophilic ligands and iron, LCN2 forms a covalently linked, disulfide-bridged heterodimer with the 92 kDa type V collagenase (MMP9). LCN2 has also been termed the 25 kDa alpha-2-microglobulin-related subunit of MMP9. The LCN2/MMP9 hetercomplex is a 125 kDa molecule.

For purposes of the present invention, the term “MMP9” refers to the MMP9 collagenase (a.k.a. CLG4B, gelatinaseB or GELB), which is a secreted zinc matrix metalloproteinase that participates in the breakdown of the extracellular matrix (ECM) in normal physiological processes including embryonic development and tissue remodeling. The MMPs are also active in disease processes characterized by remodeling including arthritis and metastasis. The reference sequence for human MMP9 preproprotein mRNA is NM_004994.2, while the reference sequence for the human MMP9 preproprotein is NP_004985. Most MMPs are secreted as inactive proproteins which are activated when cleaved by extracellular proteinases. MMP9 degrades type IV and V collagens. In vitro, Lcn2 is able to protect MMP9 from autodegradation in a dose dependant manner, thereby preserving MMP9 enzymatic activity. Yan, Li et al. “The High Molecular Weight Urinary Matrix Metalloproteinase (MMP) Activity Is a Complex of Gelatinase B/MMP-9 and Neutrophil Gelatinase-associated Lipocalin (NGAL): Modulation of MMP-9 Activity by NGAL” J. Biol. Chem., Vol. 276 (40) (2001) 37258.

As used herein, “CXCR4” refers to the C-X-C chemokine receptor type 4 (previously called fusin), which is also identified as the Cluster Designation (CD) 184 antigen. The reference sequence for human CXCR4 mRNA is NM_001008540, while the reference sequence for the human CXCR4 protein (isoform a) is NP_001008540. CXCR4 is the cell surface receptor for Stromal-Derived-Factor-1 (SDF-1), which is also known as the Chemokine (C-X-C motif) ligand 12 (CXCL12). SDF-1 is produced in two alternatively spliced forms from the same gene, SDF-1α/CXCL12a and SDF-1β/CXCL12b. All of the chemokines feature four conserved cysteines forming two disulfide bonds. In the CXC chemokines, the initial pair of cysteines is separated by an intervening amino acid. SDF-1 is strongly chemotactic for lymphocytes and is important in hematopoietic stem cell homing to the bone marrow and in hematopoietic stem cell quiescence. Drugs that block the CXCR4 receptor, such as the investigational drug AMD3100, are able to induce hematopoietic stem cell mobilization in animal and human studies. A nexus between CXCR4 and MMP9 has been experimentally identified in a lung cancer metastatic model in which it was found that inhibition of MMP9 expression inhibited SDF-la induced cell invasion and that bone marrow-derived-SDF-1a enhances the invasiveness of lung cancer cells by increasing MMP-9 expression through the CXCR4/ERK/NF-kB signal transduction pathway. Tang C-H, et al., “Involvement of matrix metalloproteinase-9 in stromal cell-derived factor-1/CXCR4 pathway of lung cancer metastasis” Carcinogenesis 29 (1) (2008) 35.

As used herein, the term “antagonist” means a molecule that does not provoke a biological response itself but binds to structurally defined sites on particular targets and thereby blocks at least one of the biological activities of the target. Antagonists include but are not limited to antibodies, engineered receptor ligands, and other structure based binding entities such as aptamers. The term antagonist also includes antagonists having modifications that provide for an extended biological half-life.

As used herein the term “antibody” refers to antibody like molecule that binds to an antigen, and includes antibody fragments such as non-covalently linked Fab, covalently linked F(ab′)2, single domain antibodies (dAbs or VHH), Fv, scFv (single chain Fv), dsFv, and the like. Fully human or humanized antibodies, including those humanized from non-human antibodies, are included in the definition of “antibody” as well as antibody like molecules derived by phage display and other recombinant mechanisms. The term antibody also includes chemically modified antibodies such as pegylated antibodies.

As used herein the term “prophylaxis” means prevention against a future event, such as successful establishment of a colony of tumor cells that is secondary to the primary tumor. In the context of prophylaxis against tumor cell metastasis and tumor seeding that may potentially occur as a consequence of a surgical or diagnostic procedure, the prophylactic administration can occur before, contemporaneous with, and/or after the procedure.

As used herein the phrase “diagnostic mammography” means mammography performed in a patient having a suspected problem such as a lump, nipple discharge or pain. As used herein mammography includes any imaging procedure that includes breast compression. This is distinguished from screening mammography, which is conducted routinely in the absence of symptoms.

The present inventors have adopted a mechanistic approach to identify a subset of targets from the myriad of potential targets for the prevention of tumor cell metastasis and tumor seeding. Targets that have been identified as particularly critical to tumor metastasis include LCN2, LCN2/MMP9 and CXCR4. In certain embodiments, the tumor cell metastasis and tumor seeding potentially occurs as a consequence of a surgical or diagnostic procedure. Thus, in certain embodiments antagonists to the identified targets are administered before, during and/or after a procedure that might carry a risk of dissemination of tumor cells. Because the antagonists have very low toxicity, procedure related administration confers the benefit of blocking growth of any tumor cells that might be released without side effects.

Selection of LCN2 as a target for directed antagonists was derived by mechanistic identification of target interactions. One mechanism for the involvement of LCN2 in metastasis is related to LCN2 (NGAL) induced apoptosis through a receptor-mediated pathway in normal cells. Another mechanism is related to the role of LCN2 in stabilizing MMP9, a critical activity for tumor cell invasion. Screening methods to select optimal monoclonal antibodies directed to LCN2, MMP9 and the LCN2/MMP9 heterodimer that will be eventually tested in a mouse tumor model include: purified antigen binding, blocking of LCN2 receptor binding, blocking of MMP9 interaction, blocking of tumor cell invasion, and blocking of tumor cell metastasis.

In another embodiment, a mechanistic attack is directed against cancer stem cells and cancer cell interactions with tissue resident stem cells, both of which are believed by the present inventors to be important to tumor metastasis. Mesenchymal stem cells derived from bone marrow have recently been described to localize to breast carcinomas and to integrate into tumor-associated stroma. Karnoub, A. E. et al. “Mesenchymal stem cells within tumour stroma promote breast cancer metastasis. Nature, 449 (2007) 557. It has been increasingly recognized that cancer cells actively recruit stromal cells and that this recruitment is essential for the generation of a microenvironment that promotes tumor growth. Bhowmick, N. A. et al. “Stromal fibroblasts in cancer initiation and progression” Nature 432 (2004) 332. Certain of the present inventors undertook to determine the potential role of adipose tissue-derived stem cells (ASCs) in tumor growth and invasion. As discussed in more detail herein, it was determined that ASCs home to tumor sites and promote tumor growth not only when co-injected locally but also when injected intravenously. It was further demonstrated that ASCs incorporate into tumor vessels and differentiate into endothelial cells. ASCs were shown to significantly enhance the growth of murine breast cancer cells in vivo. Having shown the importance of stem cells in metastasis, the interaction between stem cells and cancer cells as mechanistically attacked as described herein.

In a mouse model the interaction between tumor cells and stem cells was found to involve SDF1 and CXCR4. SDF1 was found to be secreted by mouse stem cells and the secreted SDF1 bound to the CXCR4 receptor on murine 4T1 breast cancer cells. The SDF1 then acted in a paracrine fashion on the cancer cells to enhance their motility, invasion and metastasis. The tumor-promoting effect of ASCs was abolished by knockdown of the CXCR4 in the 4T1 tumor cells. The tumor-promoting effect of tissue-resident stem cells was also tested and validated using cells of a human breast cancer line MDA-MB-231 and human adipose tissue-derived stem cells.

The following examples are included for the sake of completeness of disclosure and to illustrate the methods of making the compositions and composites of the present invention as well as to present certain characteristics of the compositions. In no way are these examples intended to limit the scope or teaching of this disclosure.

### EXAMPLE 1

### Antagonists for Lipocalin 2

The importance of LCN2 (human NGAL/murine 24p3) in cancer progression and the desirability of blocking LCN2 for the treatment of Chronic Myeloid Leukemia (CIVIL) as well as solid tumors was disclosed by one of the present inventors (RBA) in U.S. patent application Ser. No. 11/816,011 (published as U.S. 2008/0274104, related to PCT/US06/04748, and having a first priority date of Feb. 10, 2005). Several lines of evidence have substantiated the role of LCN2 in cancer progression. Fernandez suggested that LCN2 plays an important role in breast cancer in vivo by protecting matrix metalloproteinase 9 (MMP9) from degradation. Fernández CA et al “The matrix metalloproteinase-9/neutrophil gelatinase-associated lipocalin complex plays a role in breast tumor growth and is present in the urine of breast cancer patients” Clinical Cancer Research 11(15) (2005) 5390-5395. Protection of MMP9 from degradation enhanced its enzymatic activity and facilitated angiogenesis and tumor growth. Fernandez further reported that the complex of MMP9 and NGAL is present in the urine of breast cancer patients. However, it is noted that the appearance of NGAL in the urine may not be necessarily specific for cancer diagnosis. Abbott Diagnostics has reported increased appearance of NGAL in urine in acute renal failure and is developing an assay that utilizes NGAL as a biomarker for the early diagnosis, risk stratification and prognosis of acute renal failure.

An important role of LCN2 and MMP9 in cancer generally is supported by evidence that oesophageal squamous cell carcinoma cells express up-regulated levels of LCN2 and that the enzymatic activity of the complex of Lipocalin 2 and MMP9 is much higher in oesophageal squamous cell carcinoma than in normal mucosa and correlates significantly with tumour invasion. Zhang H et al. “Upregulation of neutrophil gelatinase-associated lipocalin in oesophageal squamous cell carcinoma: significant correlation with cell differentiation and tumour invasion” Journal of Clinical Pathology 60(5) (2007) 555-561. Increased expression of LCN2 positively correlated with increased expression of MMP9 has been found in rectal cancer. Increased Lcn2 was significantly associated with depth of invasion, lymph node metastasis, venous involvement and advanced pTNM stage. Zhang X-F, et al. “Clinical significance of NGAL mRNA expression in human rectal cancer” BMC Cancer 9 (2009) 134.

Lipocalin 2 expression correlates strongly with negative steroid receptor status, neu oncogene overexpression, poor histologic grade, the presence of lymph node metastases, and a high Ki-67 proliferation index and can serve a predictor of poor prognosis in primary human breast cancer. See Bauer M et al “Neutrophil gelatinase-associated lipocalin (NGAL) is a predictor of poor prognosis in human primary breast cancer” Breast Cancer Research and Treatment 108(3) (2008) 389-397. LCN2 has subsequently been shown to induce the epithelial to mesenchymal transition (EMT) in breast cancer cells and to promote breast tumor invasion on the basis that over expression of LCN2 up-regulates mesenchymal markers, including vimentin and fibronectin, down-regulates the epithelial marker E-cadherin, and significantly increases cell motility and invasiveness. Yang J, et al. “Lipocalin 2 promotes breast cancer progression” PNAS 106(10) (2009) 3913. Similarly, in NGAL decreases E-cadherin-mediated cell-cell adhesion and increases cell motility and invasion in colon carcinoma cells. Hu, L., et al. Lab. Invest. 89 (5) (2009) 531-548.

Despite the above referenced evidence that LCN2 is elevated in certain cancers, evidence of any therapeutic effect in targeted blocking of LCN2 has been lacking. As recently reviewed, attention in the field of clinical oncology is currently focused on the potential use of NGAL levels in diagnosis, prognostication and response prediction. Boglignano, D., et al. “Neutrophil gelatinase-associated lipocalin (NGAL) in human neoplasias: A new protein enters the scene” Cancer Lett 2009.

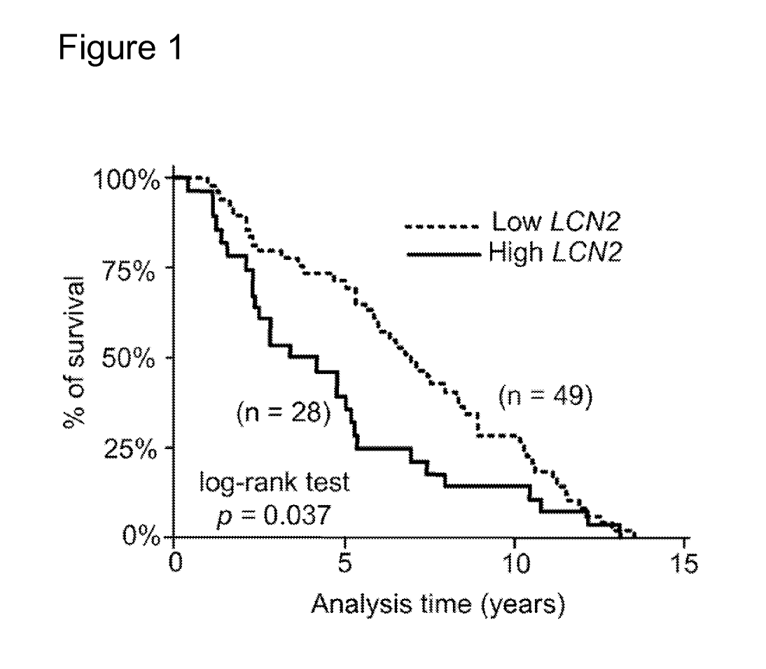

In an effort by certain of the present inventors to determine whether LCN2 is a critical determinant of tumor formation and metastasis in breast cancer, studies were undertaken by certain of the present inventors that indicate an essential role for LCN2 in breast cancer. It was found that high levels of LCN2 expression are significantly associated with two types of aggressive breast cancers, HER2 positive and triple negative breast cancers. By examining NGAL expression in 318 newly diagnosed breast cancer patients prior to any treatments (enrolled in a study in MDACC), it was found that high NGAL transcript levels are associated with poor clinicopathological features. Using a public database composed of LCN2 (the gene for human NGAL and mouse 24p3) gene expression data and survivability, certain of the present inventors found that decreased survivability is associated with high NGAL expression in breast cancer patients (FIG. 1). These data strongly suggest NGAL is a diagnostic marker, however, the further results indicate that NGAL is more than just a marker for breast cancer; rather it functions as a critical factor in enhancing breast tumor formation and metastasis.

In this regard, it was further determined that: 1) elevated LCN2results in increased tumor cell migration and invasion in vitro and tumor metastasis in vivo in mouse models, 2) deficiency of LCN2 significantly delayed mammary tumor formation and lung metastasis in MMTV-ErbB2 transgenics, 3) murine LCN2 expression is driven by HER2/PI3K/AKT/NF-κB pathway, and 4) serum levels of the LCN2/MMP9 complex and MMP9 enzymatic activity are correlated with LCN2 expression in the mouse model. Certain of these conclusions were determined as follows.

Elevated Lcn2 results in increased tumor cell migration and invasion in vitro and tumor metastasis in vivo in mouse models: Transgenic mice carrying the mutant form of ErbB2 (V664E) driven by the mammary specific promoter MMTV develop multiple primary breast tumors and lung metastases. Muller WJ, et al. “Single-step induction of mammary adenocarcinoma in transgenic mice bearing the activated c-neu oncogene” Cell 54(1) (1988) 105-15. By using MMTV-ErbB2 (V664E)^{tg/tg} mice (FVB) and mLCN2^{-/-} mice (C57B/6) (See Flo TH, et al. “Lipocalin 2 mediates an innate immune response to bacterial infection by sequestrating iron” Nature 432(7019)(2004) 917-21)), three groups of mice were generated expressing ErbB2 (V664E) with variations of mLCN2 alleles (mLCN2^{+/+}, mLCN2^{+/-}, and mLCN2^{-/-).}

The effects of these alleles were manifest in striking differences in the timing of tumor formation, as well as the number and the size of primary tumors among the three groups. Groups carrying either one or two alleles of mLCN2 started to develop multiple large (>1 cm) mammary tumors around 170 days after birth. In contrast, mLCN2^{-/-} mice did not form similar size of tumors until approximately 260 days, a time when >60% mice in the groups expressing mLCN2 were already terminated due to excessive tumor burden. The overall tumor occurrence was significantly delayed in the mLCN2^{-/-} group (260-500 days with T₅₀=303 days) compared to the mLCN2^{+/+} mice (170-340 days with T₅₀=210 days). Although the mLCN2^{+/-} group showed a slightly delayed course of tumor occurrence compared to the mLCN2^{+/+} group, no significance was observed regarding the tumor occurrence and tumor volume between these two groups, indicating that one allele mLCN2 deficiency was not sufficient to interfere with the formation of ErbB2 (V644E)-induced breast tumors. Greater numbers and larger volumes of tumor were also observed in the groups expressing mLNC2 compared to the mLCN2^{-/-} group. Notably, the lung metastases in mLCN2^{-/-} mice were significantly delayed (p<0.05) compared to the mLCN2^{+/+} mice. By Kaplan-Meier analysis, the T₅₀ for lung metastasis in the mLCN2^{+/+} group is approximately 260 days. In contrast, the T₅₀ value was not reached in the mLCN2^{+/-} and the mLCN2^{-/-} groups, suggesting that deficient mLCN2 expression also impairs lung metastasis in this model.

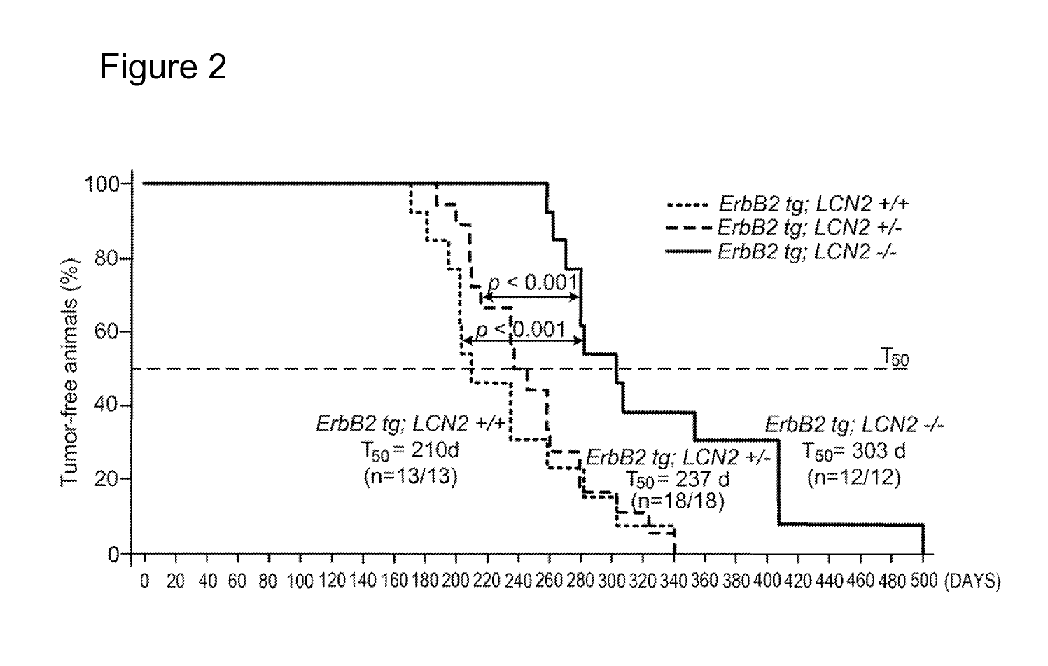

In summary it was found that mice lacking LCN2 have impaired ErbB2-induced breast cancer formation (FIG. 2). Groups carrying either one or two alleles of Lcn2 started to develop multiple large (1-2 cm) mammary tumors around 170 days after birth. In contrast, LCN2−/− mice did not form similar size of tumors until approximately 260 days, a time when >60% mice in the groups expressing LCN2 were already terminated due to excessive tumor burden. The primary tumor weight and the tumor numbers were significantly higher in mice expressing LCN2 compared to mice lacking LCN2. In addition, fewer lung metastases were present in LCN2−/− mice. Taken together, the results constitute the first genetic evidence that LCN2protein plays a key role in ErbB2-induced mammary tumor formation.

LCN2 expression in HER2⁺ breast tumor cells stimulates tumor growth and metastasis in vitro and in mouse xenograft model: Using HER2⁺ SKBr3 cells (a human tumor cell line that forms poorly differentiated adenocarcinoma in nude mice and which is characterized by high NGAL expression), a significant reduction in cell migration and invasion was observed upon knocking down LCN2 expression compared to either parental cells or cells expressing a non-specific shRNA. Cell migration and invasion assays were performed using the two-chamber migration assay (8 μm pore size, BD Biosciences). For SKBr3 cells, 1×10⁵ cells were seeded in serum-free medium into the upper chamber and migrated/invaded toward 10% FCS in the lower chamber for 8 hrs. Cells migrated/invaded to the bottom of the membrane were fixed and stained with crystal violet 0.2%/methanol 20%. Quantification was performed using unpaired t-test. shRNAs were purchased from mission shRNA collections (Sigma Aldrich). NGAL shRNAs (clone D: TRCN0000060290 and clone E: TRCN0000060290) and 24p3 shRNA (TRCN0000055328) were used. Cells containing shRNA after lentiviral infection were selected with puromycin.

When mammary fat pads of nude mice were injected with one million of either parental MDA-MB-468 cells (human breast cancer cells having high LCN2 expression), or its derivatives expressing either the non-targeted shRNA or the shRNA for LCN2 42 days after implantation, primary tumor and surrounding tissues were excised and whole mammary mounts were analyzed using H&E staining. Freshly collected tissues were imaged using XENOGEN IVIS 200 Imaging System for GFP signals. For histological analysis, tissues were post-fixed with 10% neutral buffered formalin, embedded in paraffin and sectioned at 4 μm, and stained with H&E. No significant differences in the primary tumor size/weight were found among the three groups (data not shown). However, the tumor cells' capacity for invasion and metastasis, as measured by the events of lymphovascular invasion, intramammary lymph node metastasis and chest/abdominal wall invasion were significantly reduced in the group injected of MDA-MB-468 cells with the LCN2 shRNA knockdown.

LCN2 expression correlates with aggressive tumor formation in mouse mammary tumor cell lines: Using rtPCR, the association of LCN2 expression with aggressive human breast cancer types was further demonstrated when using a series of mouse breast tumor cell lines (67NR, 168FARN, 4T07, and 4T1) with distinct metastatic potentials. Aslakson CJ, Miller FR. “Selective events in the metastatic process defined by analysis of the sequential dissemination of subpopulations of a mouse mammary tumor” Cancer Res 52(6) (1992) 1399-405. Of the lines tested, 4T07 and 4T1 cells are the most aggressive and develop lung metastases. It was found that only 4T07 and 4T1 cells have LCN2 transcripts and secrete mLCN2, with the most aggressive 4T1cells having the highest levels of mLCN2. Knocking down mLCN2 by shRNA in 4T1 cells reduced MMP-9 activity and colony formation in soft agar. Similar to our findings in plasma from ErbB2-induced breast tumor-bearing mice, high levels of mLCN2 were detected in the plasma of breast tumor-bearing mice implanted with 4T1 cells and increased MMP-9 activity compared to normal healthy mice.

Murine Lcn2 expression is driven by HER2/PI3K/AKT/NF-κB pathway: LCN2 expression in SKBr3 cells was reduced in a dose-dependent manner by the PI-3K inhibitor LY294002 and by Bay11-7082, which specifically blocks IκB phosphorylation needed for NF-κB activity (FIGS. 2B-C). In addition, overexpression of dominant negative AKT led to reduced expression of LCN2, while overexpression of dominant active AKT increased LCN2 levels (FIG. 2D). These results in breast cancer are consistent with a recent study showing that LCN2 expression was largely dependent on the NF-κB pathway in thyroid neoplastic cells. Iannetti A, et al. “The neutrophil gelatinase-associated lipocalin (NGAL), a NF-kappaB-regulated gene, is a survival factor for thyroid neoplastic cells” Proc Natl Acad Sci USA 105(37) (2008) 14058.

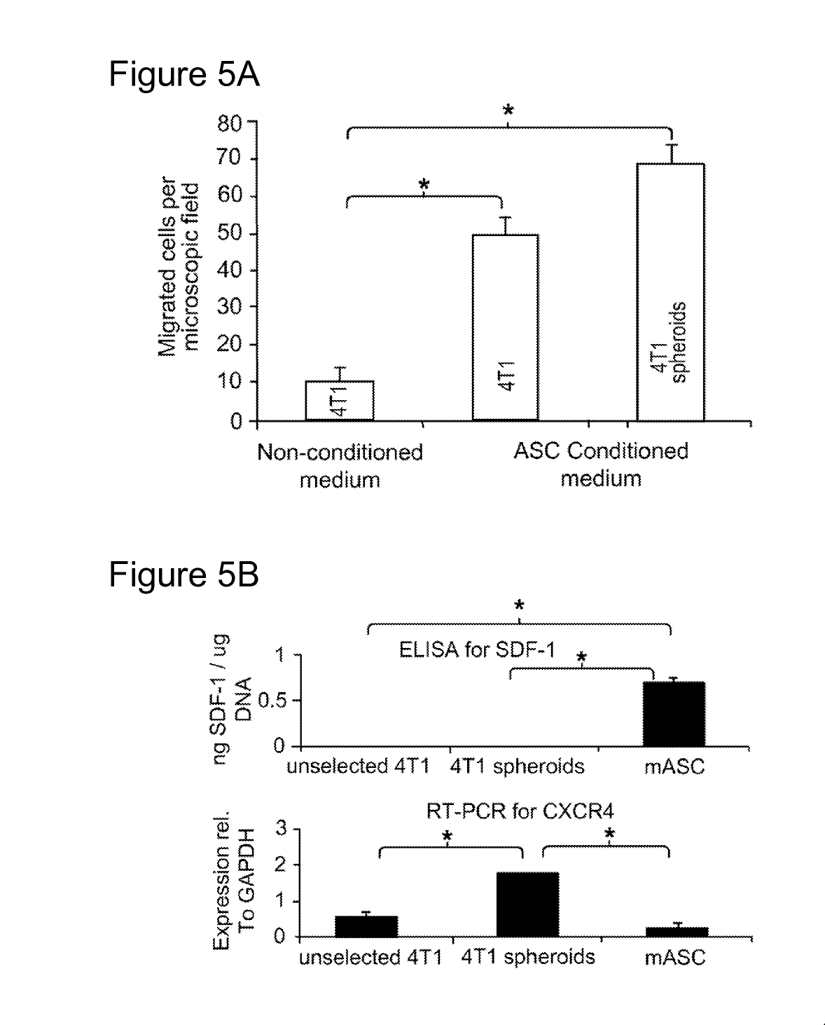

Serum levels of the Lcn2/MMP9 complex and MMP9 enzymatic activity are correlated with Lcn2 expression in the mouse model: Unlike the healthy wild-type mice with minimal level of mLCN2 (24p3) in the plasma, dramatically increased mLCN2 levels were detected in tumor-bearing MMTV-ErbB2 (V664E) mice. See FIG. 4, top panel. Interestingly, elevated MMP-9 gelatinase activity and the presence of higher molecular weight gelatinase activity was noted in the plasma of tumor-bearing mice expressing LCN2 compared to mLCN2^{-/-} group and normal healthy mice. FIG. 4, bottom panel. The decreased and fragmented MMP-9 gelatinase activity was also noted in the plasma of tumor-bearing mLCN2^{-/-} mice, indicating the function of LCN2 in maintaining MMP-9 activity and stability.

Reduction in Metastasis by Blocking LCN2: In exploring the possibility that inhibiting secreted LCN2 could block distant tissue metastasis, it was determined that i.v. injection of antibody against LCN2 virtually blocked lung metastasis after breast tumors had been formed in the mouse mammary tumor (4T1) model. In these studies an affinity purified rabbit polyclonal antibody was generated against mouse LCN2 using recombinant proteins purified from E. coli as an antigen. Goetz DH, et al. “The neutrophil lipocalin NGAL is a bacteriostatic agent that interferes with siderophore-mediated iron acquisition” Mol Cell 10(5)(2002) 1033-43. The antibody was intravenously into nude mice seven days after implantation with 5000 GFP-labeled 4T1 mouse breast cancer cells, when visible breast tumors were already formed (˜2 mm). Antibody injection (˜100 μg) was done once a week for a total of four times. A dramatic decrease in lung metastasis was observed in the anti-LCN2-treated group as compared to the control IgG-treated group, as measured by GFP signal intensities and pathology analyses in freshly collected lung tissues. No obvious toxic effects were noted. Histological examination confirmed significant differences in lung metastases between the two groups. The levels of circulating 4T1 tumor cells were found to be reduced in the blood of anti-mLCN2 antibody treated mice, suggesting that the antibody not only affected circulating LCN2 protein function but also reduced the number of tumor cells. Taken together, the results strongly suggest that NGAL could serve as a new therapeutic target for treating breast cancer metastasis.

### EXAMPLE 2

### Antagonists for CXCR4

The cancer stem cell hypothesis has two separate but related components. The first argues that normal stem cells become oncogenically transformed. The second suggests that oncogenically transformed cells take on some but not all stem cell properties. In either situation, these stem cell-like cancer cells are believed by the present inventors to be an active subset of the cancerous tumor that disseminates the tumor throughout the cancer patient leading to a lethal malignancy. As described in more detail below, certain of the present inventors have discovered in an in vitro model that breast cancer cells and adipose tissue stem cells (ASCs) interact in cell culture and that these cell mixtures have increased tumor formation and metastases compared to the breast cancer cells alone. It has been further established that CXCR4 plays a role in this interaction and that targeting CXCR4 reduces the potential for metastatic spread.

4T1 Mammosphere Model: A model of the aggressive phenotype of cancer cells capable of metastasis was first necessary such that potentially viable inhibitors could be identified. Spheroid-formation has been identified as one feature of metastasis initiating cells. To enrich the small subpopulation of such metastasis initiating cancer cells, a selection method was developed by culturing 4T1 murine mammary tumor cells in a special serum-free medium containing bFGF/epidermal growth factor/leukemia inhibiting factor for 7 days. Specifically, 4T1 cells were purchased from American Type Culture Collection (ATCC, Manassas, Va.) and cultured in RPMI 1640 medium from Cellgro (Manassas, Va) supplemented with 10% heat-inactivated fetal bovine serum (FBS) (Atlanta Biologicals, Lawrenceville, Ga.), 2 mM glutamine, 100 U/ml penicillin and 100 ug/ml streptomycin. Spheroid forming 4T1 cells (termed “4T1 mammospheres”) were selected by culturing in a defined serum-free medium (Dulbecco' s modified Eagle's medium—F12) supplemented with 2% B-27 (Invitrogen, Carlsbad, Calif.), 40 ng/ml recombinant human fibroblast growth factor basic (bFGF) (Chemicon, Billerica, Mass.), 100 ng/ml thrombin (R&D Systems, Minneapolis, Minn.), 20 ng/ml epidermal growth factor, 1 mM 2-mercaptoethanol, 1 ng/ml leukemia inhibiting factor and 1% insulin transferin sodium selenite (ITS) (Sigma, St. Louis, Mo.). The enrichment medium allows selection of spheroids of cancer progenitor cells (also called cancer-initiating or cancer stem cells) while depressing the growth of regular cancer cells. After 24 hours of culture, nonadherent or only partially adherent cells are noted. Large spheroids are apparent after 72 hours. Normally, at least 4000 regular 4T1 cells are required to induce tumors in mice. However, the injection of only 100 spheroid-derived cells into eight nude Balb/c mice resulted in tumor formation in all cases (data not shown) indicating a remarkable tumor initiation potential and thereby providing a model of metastatic spread.

Isolation of ASC: Perirenal, pelvine and subcutaneous fat tissue were dissected from 10 Balb/c mice, washed in phosphate buffered saline and immediately processed. After mincing the tissue into 2 mm³ pieces, serum-free a-modified Eagle medium (1 ml/1 g tissue) and 2 U/g tissue Liberase Blendzyme 3 (Roche Diagnostics, Indianapolis, Ind.) was added and incubated under continuous shaking at 37° C. for 45 min. The digested tissue was sequentially filtered through 100 and 40 μm filters (Fisher Scientific, Pittsburgh, Pa.) and centrifuged at 450 g for 10 min. The supernatant containing adipocytes and debris was discarded, and the pelleted cells were washed twice with Hanks' balanced salt solution (Cellgro) and finally resuspended in growth media containing alpha-modification of Eagle's medium (Cellgro), 20% FBS (Atlanta Biologicals), 2 mM glutamine (Cellgro) and 100 U/ml penicillin with 100 μg/ml streptomycin (Cellgro). Plastic-adherent cells were then grown in Nunclon culture vials (Nunc, Rochester, N.Y.) at 37° C. in a humidified atmosphere containing 5% CO₂ followed by daily washes to remove red blood cells and non-attached cells. After 80% confluence of passage 0, cells were seeded at a density of 3000 cells/cm, (passage 1). ASCs so isolated are positive for CD44 (99.36±0.75%), CD90 (97.59±2.45%) and CD105 (98.51±1.83%) and negative for CD11b (0.33±0.18%), CD14 (0.51±0.11%), CD34 (1.09±0.16%), CD45 (0.39±0.29%) and HLA-DR (0.68±0.92%). Clonal expansion studies have shown that these ASCs are capable of differentiating into chondrocytes, osteoblasts and adipocytes.

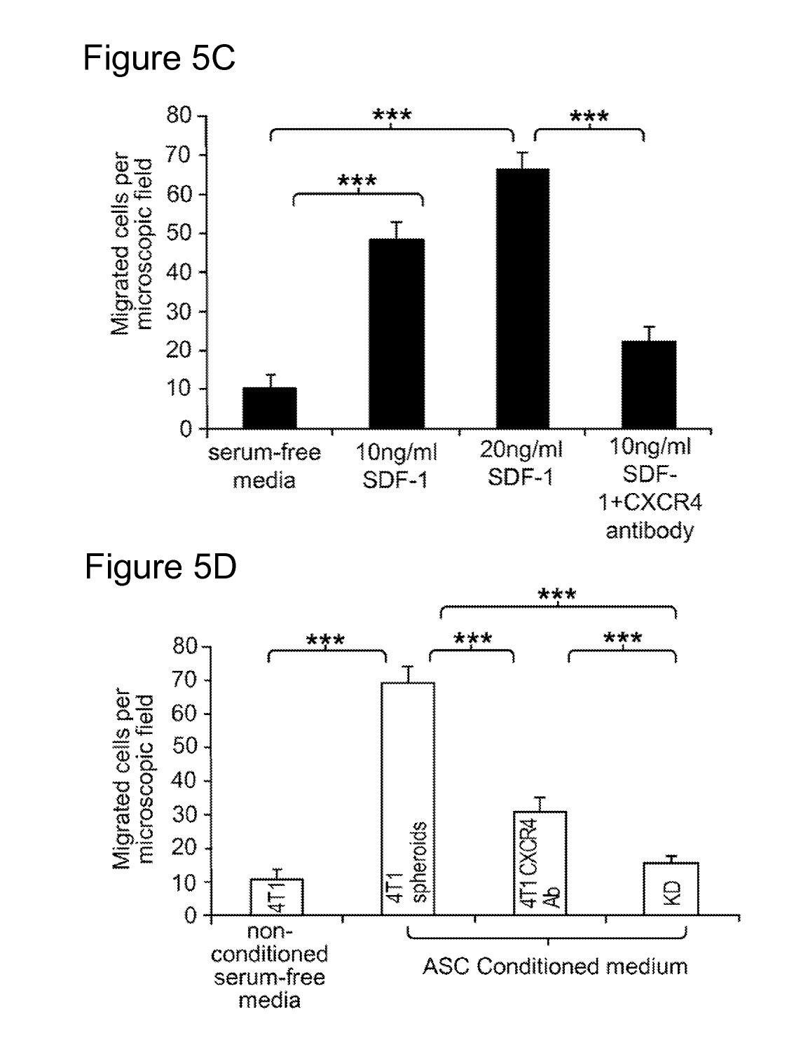

Interactions between ASC and Tumor Cells: A Fluoroblok Transwell Migration System (BD Biosciences, Bedford, Mass.) with 3 μm pore size was used for migration experiments. 4T1 cells (3×10⁴) were plated in the upper chamber of the transwell system. The lower chamber was filled with 1 ml of 72 h conditioned medium of mASC. After 24 h of migration through the transwell membrane, cells were fixed and stained with calcein. Using the in vitro transwell migration assay, it was demonstrated that 4T1 breast cancer cells migrate toward the conditioned medium of mASC (FIG. 5A). Interestingly, 4T1 mammospheres show a significantly higher number of migrating cells (˜40%) compared with unselected adherent 4T1 cells.

SDF-1 had been previously shown to play an important role in tumor growth and metastasis. Orimo, A. et al. “Stromal fibroblasts present in invasive human breast carcinomas promote tumor growth and angiogenesis through elevated SDF-1/CXCL12 secretion” Cell 121 (2005) 335-348. For blocking of potential CXCR4 mediated migration, a neutralization antibody was used (R&D Systems) at a concentration of 30 μg/ml 1 h before and during the migration assay. We found that mASC secrete elevated levels of SDF-1, whereas 4T1 cells express CXCR4 which is the receptor for SDF-1 (FIG. 5B). Interestingly again, 4T1 mammosphere cells have significantly higher messenger RNA levels of the CXCR4 receptor compared with their unselected counterparts or to mASC. Thus, a CXCR4 positive phenotype for highly malignant cells themselves emerges.

Our data showed that recombinant SDF-1 triggers migration of 4T1 spheroid-forming cells (FIG. 5C). To confirm whether the SDF-1/CXCR4 axis would be crucial for the observed differences in migration, we studied the effects by both a knockdown of CXCR4 in 4T1 mammospheres using a lentiviral short hairpin RNA construct and by receptor inhibition with a neutralizing antibody. It was found that CXCR4 short hairpin RNA substantially suppressed CXCR4 protein expression by western blot analysis. Both the CXCR4 knockdown (KD) and receptor neutralization led to a significant decrease in migration of 4T1 mammospheres toward mASC conditioned medium suggesting a pivotal role of SDF-1 in initiation of this migratory effect (FIG. 5D).

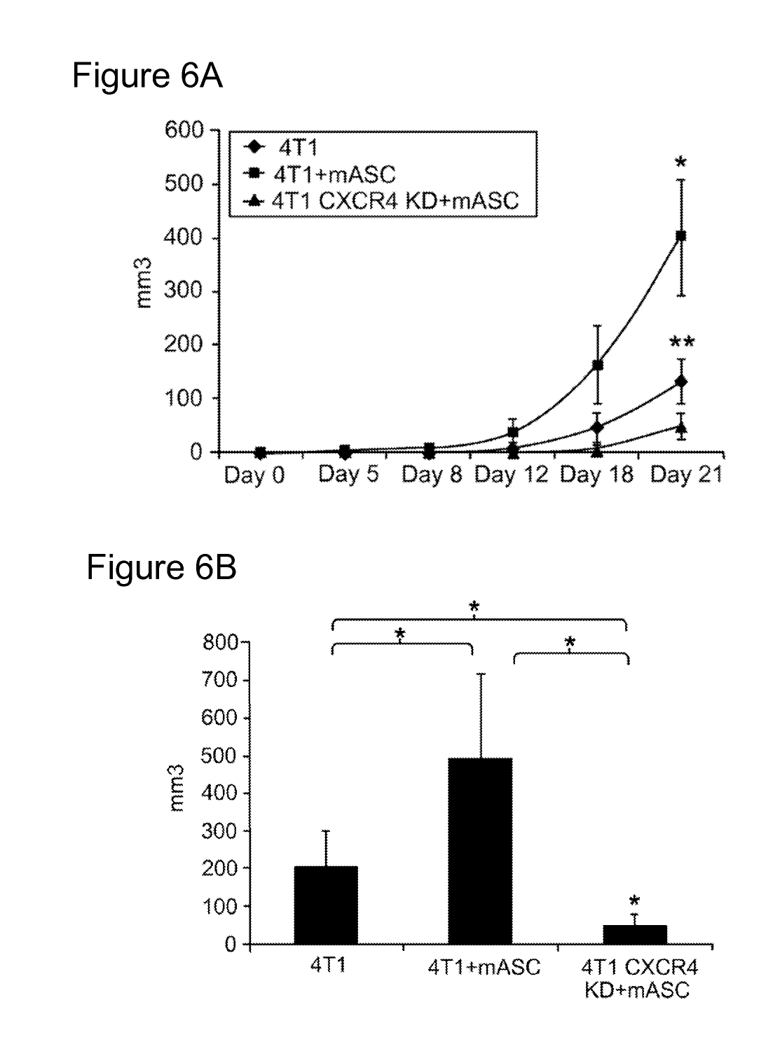

Promotion of metastatic potential by ASC was importantly shown in vivo as follows. A quantity of 5×10³ 4T1 mammospheres were injected subcutaneously into the mammary fat pad in each of ten mice in one test group and compared with injection of 5×10³ 4T1 mammospheres together with 5×10⁴ GFP-labeled mASC in another group. Tumor volumes were measured by scientific caliper. After 3 weeks, microCT scans were performed in order to determine exact final tumor volumes and to locate metastasis. Tumors in mice injected with 4T1 mammospheres and mASC formed a macroscopic visual tumor earlier (10/10 five days after injection) than the group only injected with 4T1 mammospheres (6/10 five days after injection and 10/10 after 12 days). Tumor growth rates were also significantly higher in the co-injection group (0.44 mm/day average increase in diameter) compared with injected with mammospheres alone (0.3 mm/day average increase in diameter). At the time of microCT scans (day 21 post-injection), the average tumor volume of mice injected with 4T1 mammospheres together with mASC was 490.8 mm³ (±225 mm³), whereas tumors in the 4T1 group without ASCs only measured 202 mm³ (±98 mm³). In order to investigate if the in vitro findings of SDF-1 serving as a chemoattractant for 4T1 cells would be of relevance in vivo, co-injection of 5×10⁴ mASC and 5×10³ 4T1 mammospheres subject to CXCR4 knockdown (by transfection with a third generation lentivirus containing short hairpin RNA construct against CXCR4). As shown in FIG. 6A, the tumors formed in this group developed significantly later (0/10 at day 5, 2/10 at day 8 and 8/10 at day 12) and grew slower with a 0.21 mm/day average increase in diameter compared with the other groups. On necropsy, the average tumor size was 47 mm³ (±28.8 mm³) in the CXCR4 knock-down treated mice. This represents a 76.7 and 90.4% reduction in tumor size compared with 4T1-alone group and 4T1 plus mASC group, respectively (FIG. 6B).

Metastasis into the lungs was found to be blocked by CXCR4 knockdown and increased when mASC are co-injected with 4T1 mammospheres. High-resolution microCT images of the thoracic, abdominal and pelvine body parts of the mice were diligently analyzed in order to detect metastasis, especially in the lungs. In the 4T1 group without ASCs (n=10), three mice were free of lung metastases and only moderate numbers of lung metastases were found in the remaining mice. In contrast, 9 of the 10 mice in the stem cell coinjection group showed multiple lung metastases. Only four mice in the knockdown group (n=9) showed single small-sized metastasis in the lung, suggesting that the SDF-1/CXCR4 axis is essential not only to the growth of the primary tumor but also for its ability to metastasize.

In order to find out whether ASCs produce more SDF-1 under the influence of the tumor microenvironment, 1.5×10⁴ GFP-labeled 4T1 mammospheres were injected into the mammary fat pad of five nude Balb/c mice. Another five mice have been injected with 20 μl phosphate-buffered saline as a control group. After 2 weeks, tumors have formed in mice injected with 4T1 mammospheres (average tumor diameter 7.2 mm±0.98 mm), mice were killed and the mASCs were isolated from fat tissue surrounding the tumor. mASC isolated from these cancer mice showed an increase of SDF-1 release. In further studies it was determined that ASCs could differentiate into cancer-associated fibroblasts or myofibroblast, we dissected out the tumors forming in 4T1 mammosphere injected mice and prepared single-cell suspension and quantified SMA-positive cells. It was found that ASCs express 42.47±1.42% SMA before injection and this number increased to 57.03±3.01% in ASCs isolated from tumors (P<0.01). Tumor growth in the CXCR4 knockdown group was partially encapsulated and resembled symmetrical spherical growth with an exact tumor margin, whereas the 4T1 group and the stem cell group plus 4T1 showed distorted spherical symmetries, invasive growth patterns, coarse tumor margins and satellite structures. Hematoxylin and eosin staining of tumor margins. The invasive nature of 4T1 tumor cells plus ASC was confirmed in a Boyden chamber.

Vascularity and capillary density are also enhanced by mASC interactions with tumor cells while the 4T1 CXCR4 Knockout+mASC group showed lower enhancement and accordingly a decreased vascularity of the corresponding tissue. The data also showed that ASCs are incorporated into tumor vessels and some of them colocalized with vWF staining indicating differentiation of mASCs into endothelial cells (ECs).

Vascular endothelial growth factor (VEGF) as been considered to have an important role in tumor-induced neoangiogenesis. It was demonstrated 4T1 spheroid-forming cells secrete high levels of VEGF (104.8±7.4 pg/10⁶ cells/24 h) that is significantly reduced in 4T1 spheroid-forming cells with CXCR4 knockdown (46.4±2.53 pg/10⁶ cells/24 h).

It was further remarkably demonstrated that systemically delivered mASC home to tumor sites and promote tumor growth. In making this determination, 3×10⁵ GFP-labeled mASC were injected into the tail veins of eight nude Balb/c mice that had been injected with 3×10⁴ 4T1 mammospheres 12 h before mASC injections. Subcutaneous injections of 3×10⁴ 4T1 mammospheres alone and coinjection of 3×10⁴ 4T1 spheroid-forming cells together with 3×10⁵ GFP-labeled mASC served as control groups. Tumor growth in mice injected with mASC intravenously (i.v.) was enhanced (398±103 mm³) compared with tumors without mASC injections (198±20 mm³) (P<0.03) suggesting a supportive effect of mASC following the delivery into the circulatory system. However, mASC directly co-injected with 4T1 cells augmented tumor growth even more (698±60 mm³). It was calculated that approximately one-fifth of i.v. injected ASCs (19.4±2.5%) are found at the tumor site suggesting that an active recruitment of ASC.

The murine 4T1 models data was confirmed using the MDA-MB-231 human breast cancer model in which 5×10⁴ MDA-MB 231 cells were injected into a group of 10 nude Balb/c mice and 5×10⁴ MDA-MB 231 cells plus 5×10⁵ human ASC into another group of 10 mice subcutaneously into the inguinal mammary fat pad. There was no tumor formation observable in the group injected with MDA-MB-231 cells alone after three months; whereas six mice in the group co-injected with ASC showed tumor formation within 30 days suggesting that the tumor promoting effect of tissue-resident stem cells is a general phenomenon.

The findings indicate that the interaction of local tissue-resident stem cells with tumor stem cells plays an important role in tumor growth and metastasis and identifies CXCR4 as a select target for inhibiting metastasis. Antagonists against CXCR4 are thus employed in a unique prophylaxis against tumor seeding and metastasis associated with surgical and diagnostic procedures.

All publications, patents and patent applications cited herein are hereby incorporated by reference as if set forth in their entirety herein. While this invention has been described with reference to illustrative embodiments, this description is not intended to be construed in a limiting sense. Various modifications and combinations of illustrative embodiments, as well as other embodiments of the invention, will be apparent to persons skilled in the art upon reference to the description. It is therefore intended that the appended claims encompass such modifications and enhancements.

1. A method of inhibiting formation of tumor metastases in a patient having a surgical or diagnostic procedure that carries a risk of dissemination of tumor cells, comprising prophylactic administration of an antagonist to CXCR4, wherein the antagonist is administered before, contemporaneous with, and/or immediately after the surgical or diagnostic procedure and the administration limits tumor cell migration and metastasis to a further site as a consequence of the surgical or diagnostic procedure.

2. The method of claim 1, wherein the antagonist is an antibody.

3. The method of claim 1, wherein the antagonist is an aptamer.

4. The method according to claim 1, wherein the prophylactic administration of the antagonist is carried out in preparation for a surgical procedure to remove tumorous material.

5. The method according to claim 1, wherein the prophylactic administration of the antagonist is carried out in preparation for a biopsy procedure.

6. The method according to claim 1, wherein the prophylactic administration of the antagonist is carried out in preparation for a diagnostic mammogram.

7. The method according to claim 1, wherein the prophylactic administration of the antagonist is carried out in conjunction with a lumpectomy.

8. The method of claim 1, wherein the prophylactic administration includes a series of administrations sufficient to maintain an effective level of the antagonist in the patient until such time as any tumor cells disseminated by the procedure have lost a potential to colonize a secondary site in the patient.

9. The method of claim 1, wherein the prophylactic administration is performed within a time frame of one week prior to the surgical or diagnostic procedure to one month after the surgical or diagnostic procedure.

10-13. (canceled)

14. A method of reducing tumor metastasis in a patient following a surgical or diagnostic procedure comprising prophylactic administration of an antibody against CXCR4 concurrently with the surgical or diagnostic procedure.

15. The method of claim 14, wherein the administration includes a series of administrations sufficient to maintain an effective level of the antibody in the patient until such time that any tumor cells disseminated by the procedure have lost a potential to colonize a secondary site in the patient.
